(19) United States
(12) Patent Application Publication
(10) Pub. No.: US 2006/0188579 A1
(43) Pub. Date: Aug. 24, 2006

(54) Novel process

(76) Inventor: Philippe Rogueda, Leicester (GB)

Correspondence Address: FISH & RICHARDSON P.C., P.O BOX 1022, MINNEAPOLIS, MN, 55440-1022, US

(21) Appl. No.: 10/563,685

(22) PCT Filed: Jul. 5, 2004
(86) PCT No.: PCT/GB04/02882

§ 371 Date: Jan. 6, 2006

(30) Foreign Application Priority Data
Jul. 7, 2003 (SE) 0302029-4

(51) Int. Cl. A61K 9/14; B29B 9/00

(52) U.S. Cl. 424/489; 264/5; 514/179

(57) ABSTRACT

The invention relates to a novel procedure for the production of a high yield of small crystalline particles of a narrow size distribution.

This invention relates to a novel procedure for a high yield production of small crystalline particles of a narrow size distribution. These particles are especially useful for therapeutic use via parenteral and inhalation routes. This invention is both easy to perform, efficient and does not require specialist equipment. It involves the dissolution of a compound into a suitable solvent and precipitation of the particles from solution using a miscible precipitant that is being sonicated.

### 1. INTRODUCTION

The control of particle size and crystallinity are important for all dosage formulations. Both of them affect the therapeutic potential, stability of the product (e.g. aggregation) and manufacturing processes (e.g. flow properties).

Crystallinity affects the stability of particles. Production of amorphous particles can result in unstable formulations, which over time can revert back to a more stable crystalline form, making them potentially unsuitable for the intended use. Such occurrence would alter the physical characteristics of both the drug particle and the formulation as a whole. The ‘shelf-life’ of such a product would greatly depend on the stability of the polymorph being used; hence it would be ideal to produce particles of the most stable crystalline nature ensuring optimum stability and the longest shelf life.

Particle size is also a significant matter for pharmaceutical applications. Control of particle size in suspensions is important for stability purposes, as the degree of flocculation and aggregation depend on it. For inhaled drug therapy there is a very specific narrow size range that must be met to avoid early deposition, and ensure penetration into the lower respiratory tract.

Inhaled drug therapy, via both the oral and nasal route, is recognized for its importance in both localised drug delivery to the lungs and for systemic applications. The respiratory tract has a whole range of in-built defences to prevent entry of external substances that can potentially be pathogenic. The reason for this is the minimal protection present in the deep lungs (respiratory ducts and alveoli). Hence, for a drug to be used for inhalation therapy, in addition to the requirements applied to all pharmaceuticals, it also needs to overcome these intrinsic defences to ensure efficient delivery. Larger particles are often removed prematurely, mainly by early impaction and sedimentation, resulting in a low availability at their site of action Furthermore, very small particles are either removed during normal breathing movements (as they are too small for diffusion, and deposition on the lung tissue), or tend to form large masses due to aggregation and agglomeration.

An aerodynamic diameter of less than 5 μm is generally considered to be appropriate for inhalation therapy. However it is now widely accepted that the ideal size range to avoid early impaction and sedimentation is far below this value. Studies carried out on inhaled drug therapy have now demonstrated that the ideal particle size range is between 0.5-5 μm. The data presented by Lippmann et al.¹ indicates that maximal deposition in the lower respiratory tract is achieved with a size range of between 2.5-3 μm. Thus particles for inhalation therapy are generally required to have an aerodynamic diameter of between 1 to 10 μm, particularly 1 to 5 μm and especially 1 to 3 μm.

The most common formulations used for inhalation therapy include both hydrophilic (such as salmeterol and formoterol) and hydrophobic compounds (such as budesonide). The latter example is a potent glucocorticoid, which is widely used in the treatment of respiratory diseases such as asthma and chronic bronchitis. Its mode of action is to reduce local inflammation by binding onto steroid receptor elements within the cell nucleus—with the overall effect to inhibit the onset of inflammation. Due to both the site of its receptors, and its response dependent on proteins produced within the nucleus, the effects of budesonide have a long onset of action but also a prolonged duration. Formoterol is also a long-acting drug in the treatment of asthma, but has a rapid onset. It is a mildly selective β₂-adrenoceptor agonist, which acts on smooth muscle receptors located on cells lining the inner walls of lower respiratory tract. Production of very small particles would result in very deep penetration. It will also ensure that a greater proportion reaches the primary site of interest (the bronchi walls).

Thus there is a requirement in the pharmaceutical industry to produce small crystalline particles of a narrow size distribution. The current techniques used often involve particle size reduction of crystals precipitated out from solution. These crystallised particles tend to be large, to have non-uniform shapes and distributions, and require further processing before use. Milling and micronisation are the techniques of choice. Both employ a great deal of mechanical energy to reduce the size of larger particles, by the processes of communition and attrition. Ideally, large crystals would be fragmented into a uniform distribution of smaller crystalline particles. However, mechanical processing can deform particles, and subsequently alter their crystal habit and morphology i.e. affect stability. Furthermore these processes are known to pose contamination issues, to produce low yields, to yield primarily amorphous material, and the subsequent high input of mechanical energy can result in the build-up of electrostatic charges promoting particular aggregation over time.

### 2. BACKGROUND

Salting out precipitation (i.e. addition of a miscible non-solvent to a drug solution) often produces crystalline particles, avoiding all the drawbacks of mechanical particle size reduction previously mentioned. However, the efficient control of particle size has always been the difficult in preventing its use in industrial applications.

The application of sonic energy to a liquid medium results in the generation of gas voids (a process known as cavitation). These ‘bubbles’ are thought to act as sites of nucleation for crystals. Furthermore, their subsequent collapse (known as implosion) creates shear forces, which can cause the fragmentation of larger crystals. Therefore sonic energy applied during precipitation can control and reduce particle size.

The use of sonocrystallisation can eliminate the need of size reduction after crystal formation, thus removing a step in the manufacturing process, and increasing the yield by preventing loss, saving both money and time.

We have now invented a novel way of crystallising small particles by specifying the ideal conditions to control particle size and crystallinity for the production of pharmaceutical substances, which has a high yield, is reproducible and can be used easily.

Previous studies have been unable to produce particles of such a small diameter, narrow distribution and crystalline nature. We have devised a simple method of precipitation, which can be performed in an open container such as a beaker, without the use of specialist equipment. Furthermore, we have optimised the crystallisation procedure, and are now able to specify the ideal conditions to produce particles within a given size range. With respect to inhalation therapy, we are able to define the ideal conditions to produce crystalline particles within 0.5-5 μm for hydrophobic drugs, and between 1-10 μm for hydrophilic drugs.

U.S. Pat. No. 6,221,398 B1 describes a procedure involving the crystallisation of inhalable drugs by the addition of a drug solution to a non-solvent. The particles produced are claimed to be smaller than 10 μm. However, the procedures employed involve the use of specialist mixing equipment (e.g. ‘ultraturrax’, and ‘ystral’). The method proposed in our work merely uses an optional magnetic stirrer, which could be removed due to the mixing effect of sonication. The procedure mentioned produces particles with a d_{v(0.9)} lower than 5.7 μm, if the slurry produced is spray-dried, which in itself is a particle reduction procedure. Hence our method is both superior in being simpler, and not requiring further treatment.

International patent WO00/38811 describes a method for producing particles using sonic energy to produce particle below 10 μm, and most preferably between 1-3 μm. The technique employed involves the addition of a drug solution to a non-solvent, as in U.S. Pat. No. 6,221,398 B1. However, the method described utilises a complex reactor design. Our method involves a simple design of a beaker with an ultrasonic probe inserted in the liquid medium. The particle size distributions of all the drugs studied were large in comparison to the ones covered in our work. Although particles with a d_{v(0.5)} value down to 3.9 μm were produced, and down to 1.64 μm for 2,6-diamino-3-(2,3,5-trichlorophenyl)pyrazine, the size distribution is rather broad, with the lowest d_{v(0.9)} being 10.16 μm. We propose a simpler and more efficient method for which the size distribution is much narrower, with a d_{v(0.9)} value of less than 5 μm.

International patents WO02/00199 A1 and WO02/00200 A1 utilise the same complex apparatus as described in WO00/38811. The latter describes the addition of counter-ions for the precipitation of salts, and also a complex procedure to collect the crystals from the solution. The former describes a technique of separation preventing particle growth, involving distillation and freezing. The invention proposed in this application is superior, because it does not posses the flaws already mentioned from using a specialised reactor, nor does it require post processing steps.

US patent US 2003/0051659 A1, describes a process for crystallising particles with ultrasounds. The particles obtained are larger than the ones produced in our work. The sonic energy levels are not commensurate with the ones used in this work. Finally, stirring is required, which is avoided in our invention.

International patent WO99/48475 describes a process to crystallise particles in a medium with controlled viscosity. One of the way of controlling the viscosity is to use ultrasounds. However this patent does not cover the production of fine particles in the respirable range.

A study by Ruch and Matijević² suggested that budesonide crystals between 1 to 10 μm could be precipitated with the use of ultrasonic energy. However, the particles produced in their study were not of a narrow size distribution and experiments performed to reproduce their work in our laboratories indicated that the conditions employed were not the most appropriate. Experiments performed by us found that freeze-drying of the sample can actually result in particle growth. Furthermore, we have devised the ideal conditions of precipitation and altered the technique used by employing full precipitation as opposed to minimal precipitation. Example 1 demonstrates that their technique is inadequate at producing a narrow distribution of stable small crystalline particles as produced in this study.

Studies performed by McCausland and Cains^{3,4,5} from Accentus Plc. describe a novel piece of equipment, combining vortex mixing with ultrasonic energy. They have claimed to produce particles smaller than 5 μm. However their sizing was performed during precipitation, i.e. dry powder was never obtained, instead the drug slurry was sized. A secondary processing would be necessary to extract the dry powder. This is not the case in our invention. Furthermore our invention does not require complex specialist equipment to be performed.

### 3. DESCRIPTION OF THE INVENTION

According to a first aspect of the invention there is provided a process for producing micron-size crystalline particles of a drug substance that comprises mixing a solution of a drug substance to a non-solvent in a container in the presence of ultrasonic energy.

The process described in this invention is suitable for the production of pharmaceutical substances of a small and narrow size range, especially drugs and carriers for inhalation, oral (mainly suspensions) and parenteral therapies. The process of the invention has been found to be effective for producing crystalline particles with an average geometric diameter between 1-10 μm, preferably between 1-5 μm and especially between 1-3 μm.

We have found that for hydrophobic drugs the technique is able to produce yields of up to 95%, and up to 70-85% for hydrophilic drugs.

The preferred conditions for the invention have been defined and are listed below.

3.1. Type of Drugs.

The process was designed to deal with both hydrophilic and hydrophobic drugs. These could be drugs suitable for inhalation therapy, but not exclusively.

Examples of specific drugs include mometasone, ipratropium bromide, tiotropium and salts thereof, salmeterol, fluticasone propionate, beclomethasone dipropionate, reproterol, clenbuterol, rofleponide and salts, nedocromil, sodium cromoglycate, flunisolide, budesonide, formoterol fumarate dihydrate, Symbicort® (budesonide and formoterol fumarate dihydrate), terbutaline, terbutaline sulphate and base, salbutamol base and sulphate, fenoterol, 3-[2-(4-Hydroxy-2-oxo-3H-1,3-benzothiazol-7yl)ethylamino]-N-[2-[2-(4-methylphenyl)ethoxy]ethyl]propane sulphonamide, hydrochloride. All of the above compounds can be in free base form or as pharmaceutically acceptable salts as known in the art.

The invention could equally be applied to non-inhalation therapy drugs, such as oncology drugs, Iressa, and compounds for oral or parenteral therapy.

3.2. Solvents.

According to the invention suitable solvents for use with hydrophobic drugs include chloroform and alcohols, preferably ethanol and ideally methanol.

With respect to hydrophilic drugs, alcohols are the preferred solvents, more preferably short-chain alcohols such as methanol and ethanol.

3.3. Precipitants.

The precipitant (or precipitant) should be miscible with the drug solution to ensure efficient precipitation. The choice of the precipitant depends on solvent used. Suitable precipitants for hydrophobic drugs include acetonitrile and water, preferably water. Suitable precipitants for hydrophilic drugs include acetonitrile, 1,1,2,2-tetrafluoroethyl-2,2,2-triflouroethylether, diethyl ether, acetone, ethyl acetate, the most appropriate being diethyl ether and acetonitrile.

The use of HFAs as suitable solvents and precipitants is also possible. By using these, it is possible to sonocrystallise a drug directly into an aerosol formulation.

The procedure can also be used to sonocrystallise a mixture of substances from solution. This is especially useful for formulations incorporating two drugs (for combination therapies). An example of such a system includes formoterol and budesonide precipitated from an alcohol solution with the use of acetonitrile.

3.4. Volumes.

The volumes of solution and precipitant must be defined and the crystallisation performed with at least a minimal amount of precipitant to turn the solution turbid, and ideally using the maximal amount of precipitant to precipitate all the substance from solution, i.e. full precipitation (see example 2). These conditions have been summarised in table 1.

TABLE 1
volume ratios of solvents to precipitant for sonocrystallisation.

| Drug | Volume ratios (Solution:Precipitant) | | |
|---|---|---|---|
| | | Saturated in methanol | Water |
| Hydrophobic | Suggested | 10 | 3 |
| | Preferred | 3 | 8 |
| | | Saturated in methanol | Acetonitrile |
| Hydrophilic | Suggested | 2 | 11 |
| | Preferred | 1 | 15 |
| | | Saturated in methano | Diethyl ether |
| | Suggested | 1 | 1 |
| | Preferred | 1 | 13 |

3.5. Reaction Times.

For a full crystallisation to happen it is necessary to allow the reaction to continue after the addition of the drug solution to the precipitant for at least 5 minutes, preferably 15 mins and ideally above 20 minutes.

3.6. Parameters for Sonocrystallisation.

The amount of ultrasonic energy required for crystallisation in this invention is characterised by its frequency, amplitude power and burst rate.

The invention was tested with an operating frequency of 24 kHz. Frequencies in the range 20 kHz and above are deemed suitable.

The amplitude of the ultrasonic energy should between 12-260 μm, but preferably between 40-210 μm and ideally between 170-210 μm.

The total power output available from the sonic probe should be of at least 300 W/m², preferably 460 W/cm² and above.

The burst rate is the ratio between sound emission and pauses. This can be adjusted from 10% to 100% per second. The burst rate is required to be between 5% -100% (i.e. constant application), ideally between 5% to 75%.

3.7. Mixing.

A magnetic stirrer can be employed to ease the addition of the drug solution to the precipitant. The speed setting for the magnetic stirring stirrer should be altered as to prevent the formation of a vortex, as these tend to dissipate the effects of ultrasonic energy and may result in inadequate mixing.

3.8. Temperature.

For best results, the precipitation should be performed below 50° C., preferably between 5-25° C., more preferably between 5-15° C. and ideally at the lowest possible temperature at which the solvent and precipitant remain liquid, while avoiding water condensation (see example 1).

3.9. Water Content.

A small amount of water may be added to the solution of hydrophilic drugs to improve crystallisation, and to produce the smallest particles. For methanol solutions between 5 to 40% w/w of water can be added, this can be adjusted to 20% w/w when using acetonitrile as a precipitant, and 30% w/w with diethyl ether. A small amount of water or a suitable polar solvent can be added for the sonocrystallisation of hydrophilic drugs. The water content added will depend on the type of precipitant used, however it should be between 1-50% w/w, preferably between 10-40% w/w and ideally between 20-40% w/w.

3.10. Filtering.

Separation of the crystallised particles is usually carried out by vacuum filtration. The selection of the type of filter is dependent on the liquids used in the process. Membrane or fibre filters can both be used, with pore diameters of less the 0.45 μm, and preferably 0.2 μm, but ideally 0.1 μm. The preferred type of filters for precipitations involving alcohols and water is cellulose nitrate, and ideally PVDF. Processes involving alcohols and acetonitrile and diethyl ether should use PTFE or polycarbonate filters.

3.11. Growth Retardants.

The use of growth retardants such as surfactants and polymers can also be utilised to limit the size of the sonocrystallised crystals. The selection of which will be knwon by those skilled in the art, and will include cyclodextrins, polymethacrylic derivatives (e.g. Eudragit), PEG and PVP and other pharmaceutically acceptable excipients.

### 4. Experimental

4.1. Experimental Set Up.

The experimental set up used in this work consisted of an ultrasonic probe dipped into a jacketed beaker with a magnetic stirrer. The precipitant was placed in the beaker and allowed to reach equilibrium temperature. The addition of the drug solution was done with a pipette.

The ultrasonic probe used in this work was the ultrasonic processor UP 400S fitted with a S3 Micro tip sonotrode. It was purchased from Dr Hielscher GmbH (Teltow, Germany). It is a stationary ultrasonic processor with variable amplitude and cycle. The maximum amplitude being considered is 210 μm, hence with regard to the data presented, an amplitude stated as 20% will be 42 μm, and 100% will be 210 μm.

4.2. Crystallisation Process.

The correct volume of precipitant is placed inside the beaker whilst being sonicated. It is a part of this invention that sonication should be started before addition of the saturated solution. The correct volume of saturated drug solution is added with a pipette or burette. The suspension formed is sonicated for a sufficient duration of time, and then filtered to remove the drug particles. The solid particles can be placed in a freeze-drier overnight to remove any trace of solvents. It was found that particles which were fully precipitated and freeze-dried over a period greater than 12 hours did not differ in size from those which were not (see example 6).

The particles obtained are characterised by SEM (particle shape), XRPD (crystallinity) and sized.

4.3. Sizing.

Sizing of the particles was performed by laser light scattering, using the Malvern Mastersizer 2000 fitted with a 100 mm lens. 2H, 3H perfluoropentane (abbreviated to HPFP) (hydrophilic drugs) and water (hydrophobic drugs) were used as suspending media. Triton X100 was added to the liquid to provide added stability when required. The following sizing parameters were used (see table 2).

TABLE 2
parameters used for sizing with the Mastersizer 2000.

| Drug | Hydrophobic | Hydrophilic |
|---|---|---|
| Dispersant | 0.04% Triton × 100 in water | 0.04% Triton × 100 in |
| | | HPFP |
| RI of drug | 1.580 + i 0.01 | 1.61 + i 0.01 |
| RI of dispersant | 1.330 | 1.263 |

| | |
|---|---|
| Pre-dispersion | Sonicate for 10 mins |
| Obscuration | 10% to 25% |

4.4. XRPD.

XRPD was performed at ambient temperature using a Siemens D5000 X-ray powder diffractometer fitted with a scintillation detector (Bruker AXS, Congleton, Cheshire, UK). Typical conditions were: Cu Kα radiation (λ=1.5406 Å, 40 mA, 45 kV), 2-70° 2θ, divergence slit 0.5°, antiscatter slit 0.5° and receiving slit 0.2 mm. Data were usually collected using a zero background holder on which approximately 10 mg of the compound was spread thinly. The holder is made from a single crystal of silicon, cut along a non-diffracting plane and then polished to an optically flat finish. The X-rays incident upon this surface are negated by Bragg extinction. Where larger quantities of a batch were available, approximately 300 mg of sample was analysed using a standard holder.

4.5. SEM.

The morphology of the particles was investigated using a LEO430 SEM (Cambridge, UK). Prior to analysis, a small sample was mounted onto an aluminium stub using an adhesive carbon disk and sputter coated with a thin film of gold and palladium for 5 mins on a Polaron SC7640 sputter coater.

### 5. EXAMPLES

### 5.1. Example 1

### Influence of Temperature on the Crystallisation of a Hydrophobic Drug with No Sonic Energy

10 ml of a saturated methanol solution of budesonide was placed in a jacketed beaker connected to a water bath. In addition to controlling the temperature, the beaker was placed on top of a magnetic stirrer with a speed setting such as to avoid the formation of a vortex. Water was added via a burette until the solution became turbid. This was then allowed to mix for 15 mins. After filtering and freeze-drying the samples, they were analysed.

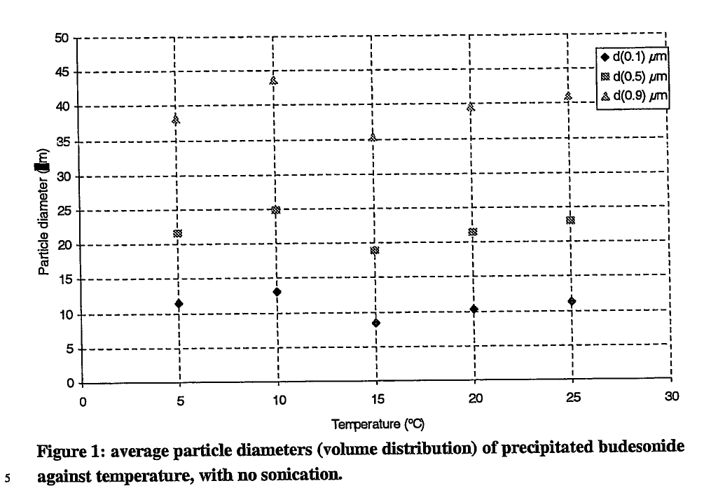

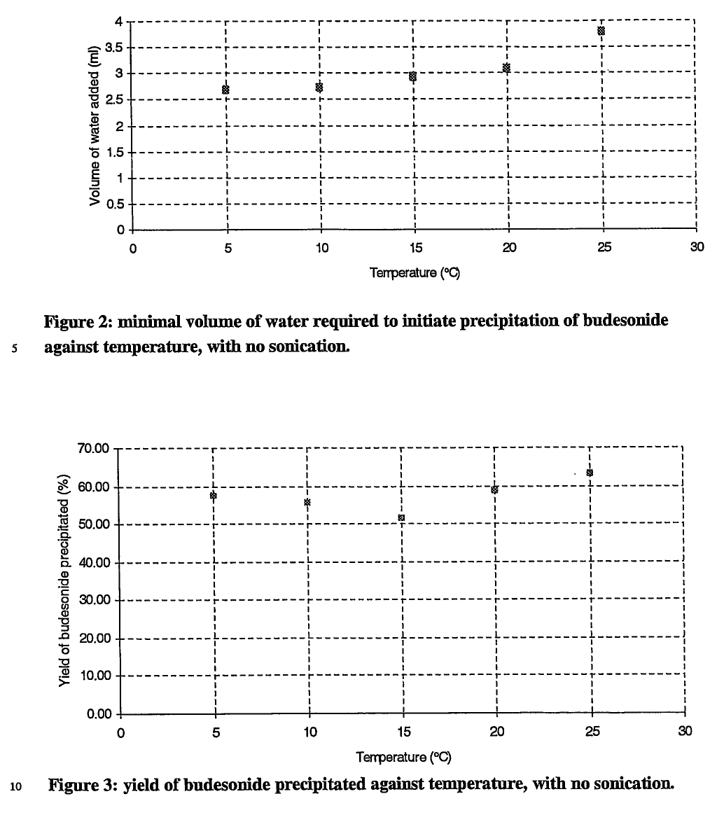

Sizing results of these particles have been summarised in table 3. FIGS. 2 and 3 show the variation of the average diameters and yield with temperature.

TABLE 3
particle diameter, yield of crystals and volume of water required for the
precipitation of budesonide at varying temperatures with no sonication.

| | Diameters | | | | |
|---|---|---|---|---|---|
| Temperature | (μm) | | | Yield | Volume of water |
| (° C.) | d_{v(0.1)} | d_{v(0.5)} | d_{v(0.9)} | (%) | (ml) |
| 5 | 11.4 | 21.6 | 38.2 | 57.5 | 2.7 |
| 10 | 13.0 | 24.8 | 43.7 | 55.7 | 2.7 |
| 15 | 8.5 | 18.8 | 35.3 | 51.5 | 2.9 |
| 20 | 10.2 | 21.4 | 39.6 | 58.9 | 3.1 |
| 25 | 11.2 | 22.9 | 41.1 | 63.3 | 3.8 |

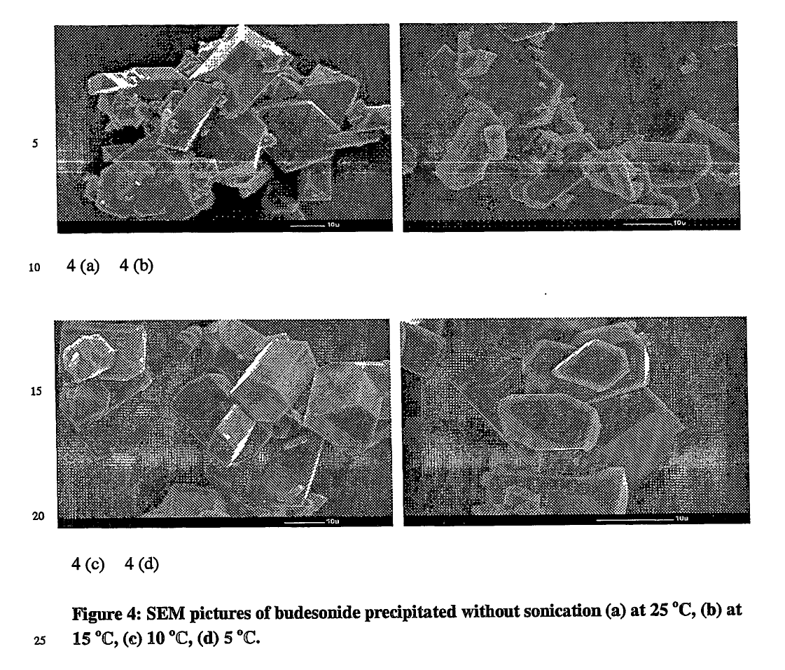

Theory suggests that a decrease in temperature results in slower crystal formation, producing smaller and more uniform shapes. However, decreasing the temperature below 15° C. does not produce smaller crystals, but slightly increases their size. The reason for this can be attributed to condensation on the sides of the beaker and the filtration unit. This could trigger the precipitation of further amounts of budesonide, and cause precipitated particles to grow (via Oswald ripening), and larger particles to form FIG. 4 illustrates this theory; it is shown that there is a decrease in the yield of budesonide from 25 to 15° C. However it increases below 15° C. although the volume of precipitant is still decreased (FIG. 3). This information also allows us to conclude that a decrease in temperature results in easier precipitation, however it does not result in earlier precipitation. If the latter were true then a decrease in the volume of precipitant would not result in a decrease in the percentage yield of budesonide from 25 to 15° C. Precipitation is slowed down as the temperature is decreased.

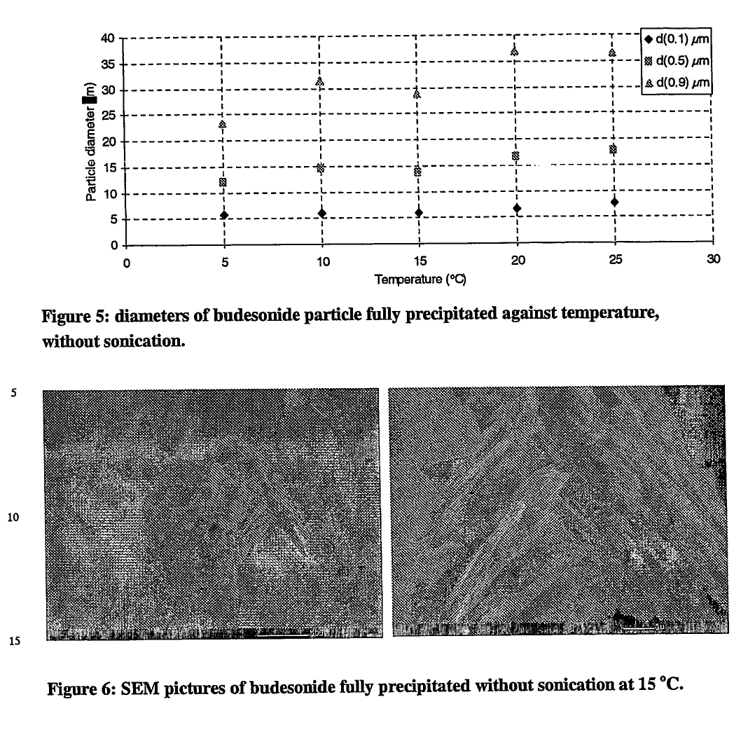

The SEM pictures of the particles produced indicate that a decrease in temperature increases the regularity of the crystal shape. FIG. 5a (25° C.) indicates that at a higher temperature crystals either cluster together, or their surface growth is predominant. Furthermore there are several smaller growths in comparison to FIG. 5d (5° C.), confirming the theory that at lower temperatures more uniform and smaller crystals are formed.

The data obtained above demonstrates that a decrease in temperature has an effect on particle diameter. The data confirms that a decrease in temperature decreases the particle size of crystals formed. Hence the ideal temperature for crystallisation is the lowest temperature possible while avoiding condensation. However the minimum amount of water required to initiate precipitation decreases with a reduction in temperature, with a plateau being reached at 5° C.

### 5.2 Example 2

### Influence of Temperature on the Crystallisation of a Hydrophobic Drug with Excess Precipitant and No Sonication

The previous study was repeated using full precipitation, i.e. adding excess water, the following results were obtained (see table 4 and FIG. 6).

TABLE 4
influence of temperature on particle diameter of budesonide particles fully
precipitated without sonication.

| Volume of water | Diameters (μm) | | |
|---|---|---|---|
| (ml) | d_{v(0.1)} | d_{v(0.5)} | d_{v(0.9)} |
| 5 | 5.75 | 12.01 | 23.26 |
| 10 | 5.91 | 14.76 | 31.37 |
| 15 | 5.94 | 13.76 | 28.83 |
| 20 | 6.69 | 16.66 | 36.81 |
| 25 | 7.61 | 17.82 | 36.45 |

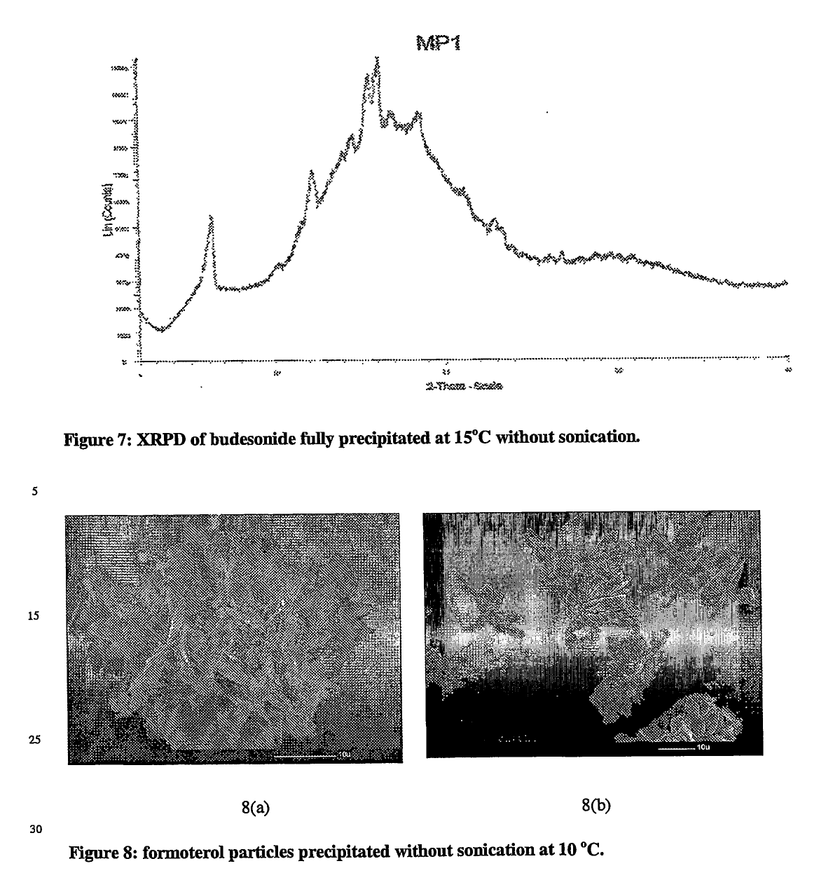

SEM pictures of the crystallised particles have been reproduced on FIG. 7. The pictures of budesonide particles fully precipitated from solution indicate that thinner clusters of sheets tend to form as opposed to octahedral crystals formed during minimal precipitation. The XRPD of these ‘sheets’ were performed and the results obtained confirm that the samples are crystalline (see FIG. 8).

The particles formed with a saturated amount of precipitants are smaller than the ones formed with a minimal amount of water. Excess precipitant helps form smaller particles.

### 5.3 Example 3

### Comparison of Crystal Characteristics Between a Hydrophobic and Hydrophilic Drug

The procedure set out in example 1 was followed. Budesonide and formoterol were precipitated without sonication under identical conditions to see their difference in crystalline shape and size. The following parameters were used whilst undertaking precipitation (table 5).

TABLE 5
precipitation conditions for comparison of particles size and shape without
sonication between a hydrophobic and a hydrophilic drug.

| | Drug | |
|---|---|---|
| | Budesonide | Formoterol |
| Solution | 10 ml saturated budesonide in | 2 ml saturated formoterol in |
| | Methanol | Methanol |
| Volume of | 2.7 ml water | 10.1 ml water |
| precipitant | | |
| Filter | 0.1 μm PVDF durapore filters | 0.2 μm PTFE filters |

| | |
|---|---|
| Temperature | 10° c. |
| Time | 15 minutes |
| Agitation | On |

The following results were obtained (table 6):

TABLE 6
comparison of particle diameters for a hydrophilic and a hydrophobic drug
crystallised from a saturated methanol solution at 10° C.,
without sonication.

| | Diameters (μm) | | |
|---|---|---|---|
| Drug | d_{v(0.1)} | d_{v(0.5)} | d_{v(0.9)} |
| Budesonide | 13.0 | 24.8 | 43.7 |
| Formoterol | 6.4 | 19.3 | 41.1 |

The results indicate that both drugs crystallise with similar size distribution, with a marginally larger diameter span for formoterol.

The SEM pictures (FIG. 9) indicate that the sample of formoterol does not consist of uniformly sized particles. Instead the pictures show that there are some very large agglomerates (or single crystals with a substantial amount of growth) along with some smaller clusters. In comparison to budesonide precipitated under the same condition (see FIG. 5c), formoterol particles are more irregular in shape.

### 5.4 Example 4

### Influence of the Volume of Precipitant on the Crystallisation of a Hydrophobic Drug

The same procedure as for example 1 was used. The experiment was performed at 15° C. The sonic probe was inserted into the drug solution prior to the addition of the precipitant (water) and switched on. The volume of water added to the budesonide solution was altered, whilst keeping the following parameters constant (see table 7).

TABLE 7
conditions for the sonocrystallisation of a hydrophobic drug.

| Conditions | | |
|---|---|---|
| Solution | | 15 ml saturated budesonide in |
| | | methanol |
| Temperature | | 15° C. |
| Time | | 15 minutes |
| Filter | | 0.1 μm PVDF durapore filters |
| Agitation | | On |
| Sonic energy | Amplitude | 100% |
| | Cycle | 0.75 |

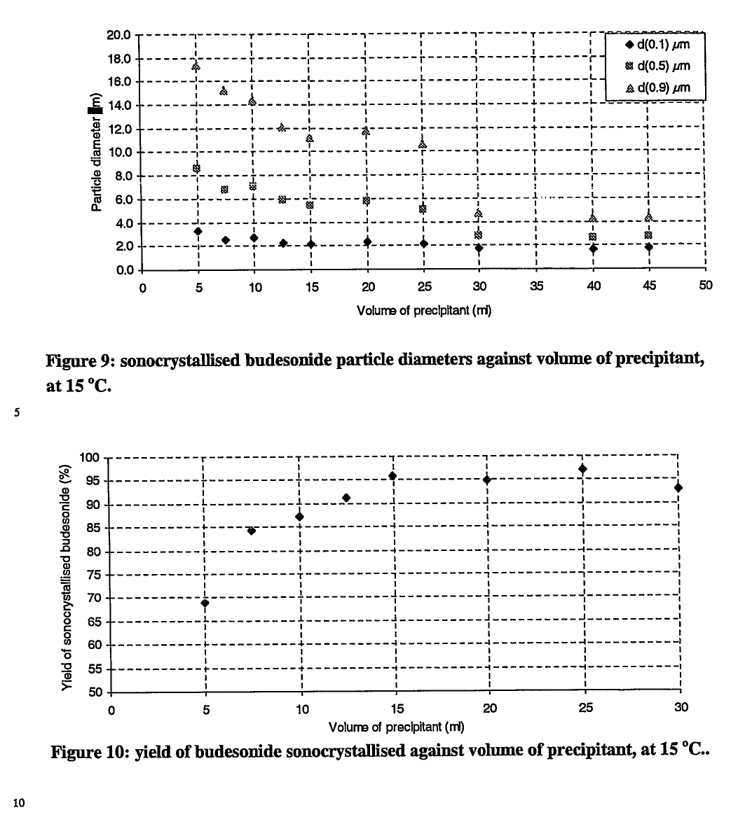

The following results were obtained (See table 8 and FIG. 10):

TABLE 8
particle diameter and yield of budesonide sonocrystallised at 15° C. from a
saturated methanol solution, whilst altering the
volume of precipitant (water).

| | Diameters | | | |
|---|---|---|---|---|
| Volume of water | (μm) | | | Yield |
| (ml) | d_{v(0.1)} | d_{v(0.5)} | d_{v(0.9)} | (%) |
| 5 | 3.32 | 8.63 | 17.4 | 69.0 |
| 7.5 | 2.48 | 6.77 | 15.3 | 84.3 |
| 10 | 2.72 | 7.02 | 14.4 | 87.2 |
| 12.5 | 2.27 | 5.89 | 12.1 | 91.3 |
| 15 | 2.10 | 5.44 | 11.2 | 95.8 |
| 20 | 2.29 | 5.82 | 11.8 | 94.9 |
| 25 | 2.15 | 5.01 | 10.6 | 96.9 |
| 30 | 1.70 | 2.80 | 4.71 | 92.8 |
| 40 | 1.63 | 2.60 | 4.23 | 69.0 |
| 45 | 1.72 | 2.73 | 4.38 | 84.3 |

This example shows that sonication reduces the size of the particles substantially. Increasing the volume of precipitant decreases the size of the particles, until a lower limit is reached.

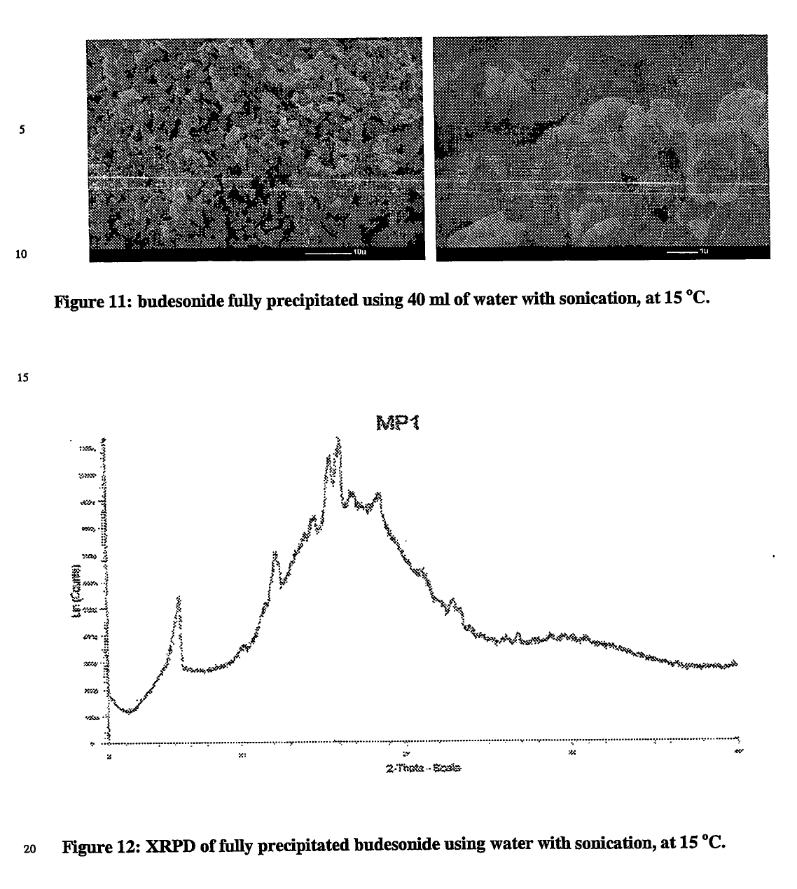

The yield of budesonide is plotted on FIG. 11, and indicates that after the addition of 25 ml of water to the 15 ml saturated budesonide solution; nearly all the drug is precipitated out.

The SEM pictures (FIG. 12) show that sonocrystallisation of fully precipitated budesonide does not result in the same crystals as for non-sonocrystallised fully precipitated budesonide (see FIG. 7).

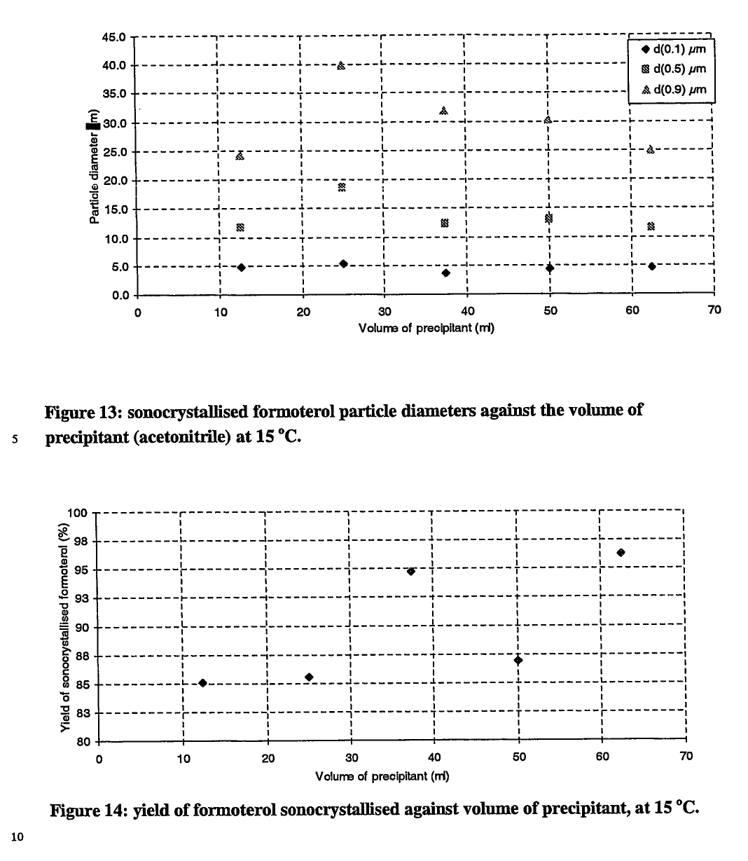

XRPD analysis (see FIG. 13) shows that the particles obtained are crystalline. In fact comparison with FIG. 8 shows that the crystals are identical.

From this example, we have found the requied ratio of water to saturated budesonide in methanol is:
for minimal precipitation: 3:10
for optimum precipitation: 8:3

### 5.5 Example 5

### Influence of the Volume of Precipitant on the Crystallisation of a Hydrophilic Drug

For experimental details see example 1, with the following amendments: a saturated solution of formoterol fumarate dihydrate in methanol was used, acetonitrile was the precipitant, and the following parameters constant were kept constant (table 9).

TABLE 9
parameters for the precipitation of a hydrophilic drug by
sonocrystallisation.

| Conditions | | |
|---|---|---|
| Solution | | 2 ml saturated formoterol in |
| | | methanol |
| Temperature | | 15° C. |
| Time | | 15 minutes |
| Filter | | 0.2 μm PTFE polypropylene |
| | | backed filters |
| Agitation | | On |
| Sonic energy | Amplitude | 100% |
| | Cycle | 0.75 |

The following results were obtained (table 10).

TABLE 10
particle diameters of formoterol sonocrystallised at 15° C. from a saturated
methanol solution, whilst altering the volume of precipitant (acetonitrile).

| | Diameters | | | |
|---|---|---|---|---|
| Volume of water | (μm) | | | Yield |
| (ml) | d_{v(0.1)} | d_{v(0.5)} | d_{v(0.9)} | (%) |
| 12.5 | 4.76 | 11.85 | 24.18 | 85.0 |
| 25.0 | 5.39 | 18.65 | 39.84 | 85.5 |
| 37.5 | 3.65 | 12.35 | 31.84 | 94.7 |
| 50.0 | 4.39 | 13.22 | 30.33 | 86.9 |
| 62.5 | 4.55 | 11.72 | 24.93 | 96.3 |

The results indicate that even if formoterol is filly precipitated from a drug solution with the use of sonic energy, large particles are still produced. Only approximately 10% of the particles lie within the ideal size range. This is further evidenced on FIG. 14.

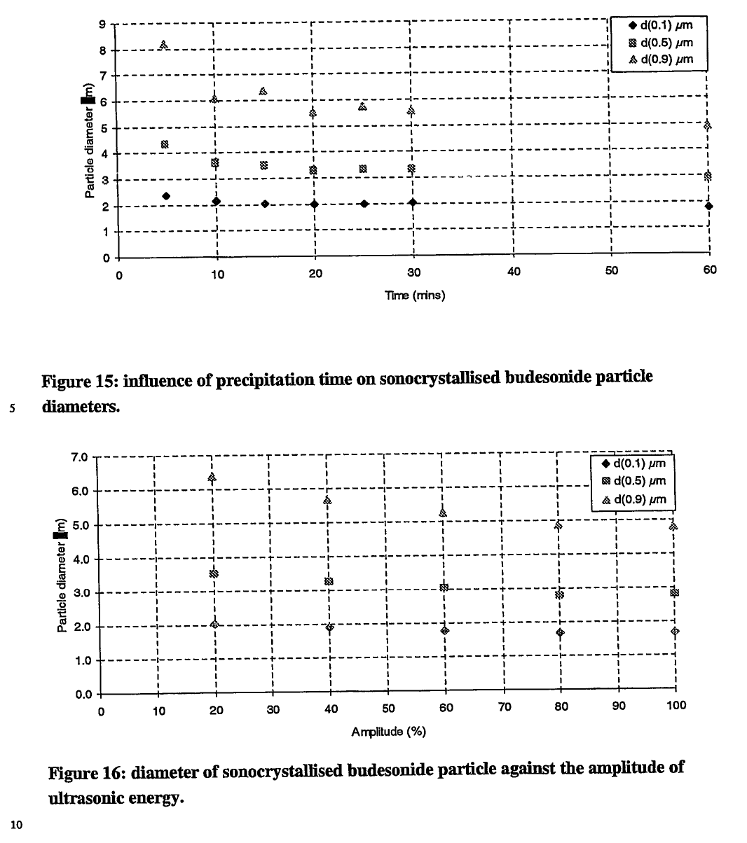

FIG. 15 shows that a yield of above 95% can be achieved. There is an unusual dip in the yield of formoterol with the volume of acetonitrile at 50 ml. This is due to filtration of the slurry. When the suspension was sized straight after precipitation (with no filtration) smaller diameters were obtained, d_{v(0.9)} value of 11.16 μm, as opposed to 30.33 μm from the powder. This indicates that crystal growth is occurring on filtration. This can be remedied by an appropriate filtration.

Smaller particles can be obtained by the addition of water, as shown further on.

### 5.6 Example 6

### Influence of Time on the Sonocrystallisation of a Hydrophobic Drug

For experimental details see example 1 with the following amendments: the drug solution was added to the precipitant while being sonicated. The time of sonocrystallisation was altered for the full precipitation of budesonide, whilst keeping the following parameters constant (table 11).

TABLE 11
parameters for the study of the influence of time on the
sonocrystallisation of budesonide.

| Conditions | | |
|---|---|---|
| Solution | | 6 ml saturated budesonide in |
| | | methanol |
| Volume of precipitant | | 16 ml water |
| Temperature | | 15° C. |
| Filter | | 0.1 μm PVDF durapore filters |
| Agitation | | On |
| Sonic energy | Amplitude | 20% |
| | Cycle | 0.25 |

The following results were obtained (table 12, FIG. 16):

TABLE 12
influence of time on the diameter of budesonide particles
sonocrystallised at 15° C. from a saturated methanol solution.

| | Diameters | | |
|---|---|---|---|
| Time | (μm) | | |
| (mins) | d_{v(0.1)} | d_{v(0.5)} | d_{v(0.9)} |
| 5 | 2.36 | 4.34 | 8.21 |
| 10 | 2.16 | 3.62 | 6.08 |
| 15 | 2.03 | 3.51 | 6.38 |
| 20 | 1.97 | 3.29 | 5.54 |
| 25 | 1.97 | 3.36 | 5.76 |
| 30 | 2.03 | 3.35 | 5.57 |
| 60 | 1.78 | 2.93 | 4.89 |

FIG. 16 shows that the particle diameter of sonocrystallised budesonide decreases wit increasing time until a plateau is reached. The greatest effect takes place between 0 to 20 minutes, after which there is only a relatively small decrease in particle diameter.

Therefore the optimum time for sonocrystallisation is above 5 minutes, preferably above 15 minutes, most preferably above 30 minutes.

### 5.7 Example 7

### Influence of the Amplitude and Cycle of Ultrasonic Energy on the Sonocrystallisation of a Hydrophobic Drug

For experimental details see example 6 with the following amendments: the volume of precipitant was kept constant whilst the amplitude of the ultrasonic probe was changed. The following parameters were kept constant (table 13).

TABLE 13
parameters for the study of the influence of the amplitude of the
ultrasonic energy on the sonocrystallisation of budesonide.

| Conditions | |
|---|---|
| Solution | 6 ml saturated budesonide in |
| | methanol |
| Volume of precipitant | 16 ml water |
| Temperature | 15° C. |
| Time | 15 minutes |
| Filter | 0.1 μm PVDF durapore filters |
| Agitation | On |

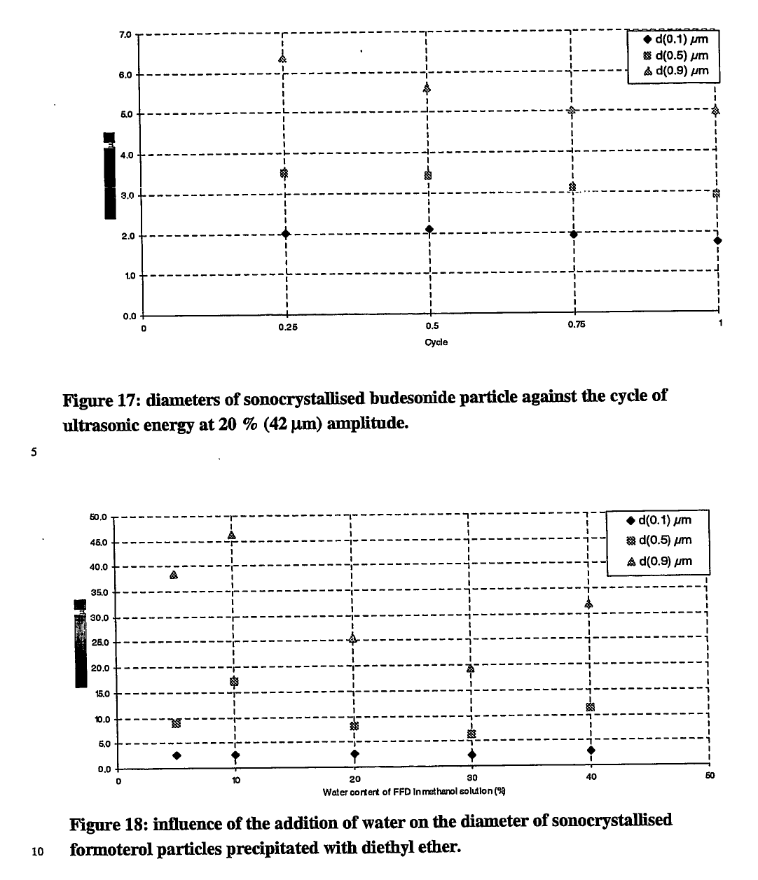

The following results were obtained (table 14, FIGS. 17 and 18).

TABLE 14
particle diameter of budesonide particles sonocrystallised
at 15° C. from a saturated methanol solution, whilst
altering the cycle and amplitude of the ultrasonic energy.

| | | Diameters (μm) | | |
|---|---|---|---|---|
| Cycle | Amplitude | d_{v(0.1)} | d_{v(0.5)} | d_{v(0.9)} |
| 0.25 | 20 | 2.03 | 3.51 | 6.38 |
| 0.25 | 40 | 1.89 | 3.25 | 5.68 |
| 0.25 | 60 | 1.77 | 3.03 | 5.26 |
| 0.25 | 80 | 1.68 | 2.78 | 4.87 |
| 0.25 | 100 | 1.67 | 2.79 | 4.80 |
| 0.50 | 20 | 2.10 | 3.42 | 5.61 |
| 0.50 | 100 | 1.42 | 2.37 | 4.08 |
| 0.75 | 20 | 1.94 | 3.11 | 5.03 |
| 0.75 | 100 | 1.45 | 2.46 | 4.32 |
| 1.00 | 20 | 1.74 | 2.92 | 4.98 |
| 1.00 | 100 | 1.88 | 3.18 | 5.41 |

FIG. 17 shows that by increasing the amplitude of the ultrasonic energy, the particle diameter decreases. The graph seems to indicate that a plateau is reached, indicating that there is a lower limit for the particle size with respect to control via amplitude alone.

FIG. 18 shows that an increase in the cycle of the ultrasonic energy also decreases particle size, with a plateau at high cycles. Particle size reduction using ultrasonic energy has a limit, after which further changes of the sonic parameters will have no effect.

The data demonstrates that the optimum parameters for sonocrystallisation arc 0.5 cycle and 100% amplitude, i.e. intermittent cycle and 210 μm.

### 5.8 Example 8

### Influence of Water Content on Sonocrystallisation of a Hydrophilic Drug

For experimental details see example 6 with the following amendments: a saturated solution of formoterol sate dihydrate in methanol with varying water content was used. The effect of water was studied with both diethyl ether and acetonitrile as precipitants. The following parameters were used (table 15).

TABLE 15
parameters for the study of the influence of water content on the
sonocrystallisation of a hydrophilic drug.

| Conditions | | |
|---|---|---|
| Solvent | | Methanol |
| Temperature | | 15° C. |
| Filter | | 0.2 μm PTFE polypropylene |
| | | backed |
| Agitation | | On |
| Sonic energy | Amplitude | 100% |
| | Cycle | 0.75 |

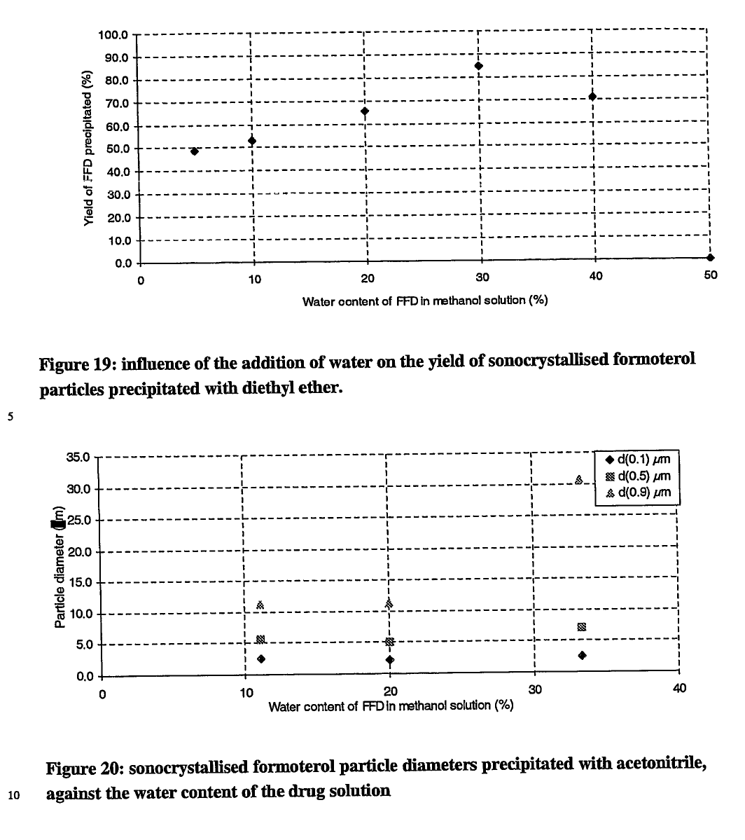

The following results were obtained (table 16, FIGS. 19 and 20).

TABLE 16
particle diameter of budesonide sonocrystallised at 15° C.
from a saturated methanol solution,
whilst altering the cycle and amplitude of the ultrasonic energy.

| | Water content | Diameters | | | |
|---|---|---|---|---|---|
| | in drug solution | (μm) | | | Yield |
| Precipitant | (%) | d_{v(0.1)} | d_{v(0.5)} | d_{v(0.9)} | (%) |
| Diethyl ether | 5 | 2.65 | 8.97 | 38.61 | 49.0 |
| | 10 | 2.57 | 17.26 | 46.28 | 53.0 |
| | 20 | 2.67 | 8.16 | 25.64 | 65.5 |
| | 30 | 2.31 | 6.44 | 19.35 | 84.7 |
| | 40 | 2.92 | 11.55 | 32.03 | 70.6 |
| Acetonitrile | 11.11 | 2.50 | 5.60 | 11.24 | 47.4 |
| | 20.00 | 2.18 | 5.13 | 11.26 | 60.9 |
| | 20.00 | 2.02 | 4.43 | 9.05 | 66.1 |
| | * temperature: 5° C. | | | | |
| | 33.33 | 2.50 | 7.01 | 30.56 | 62.4 |

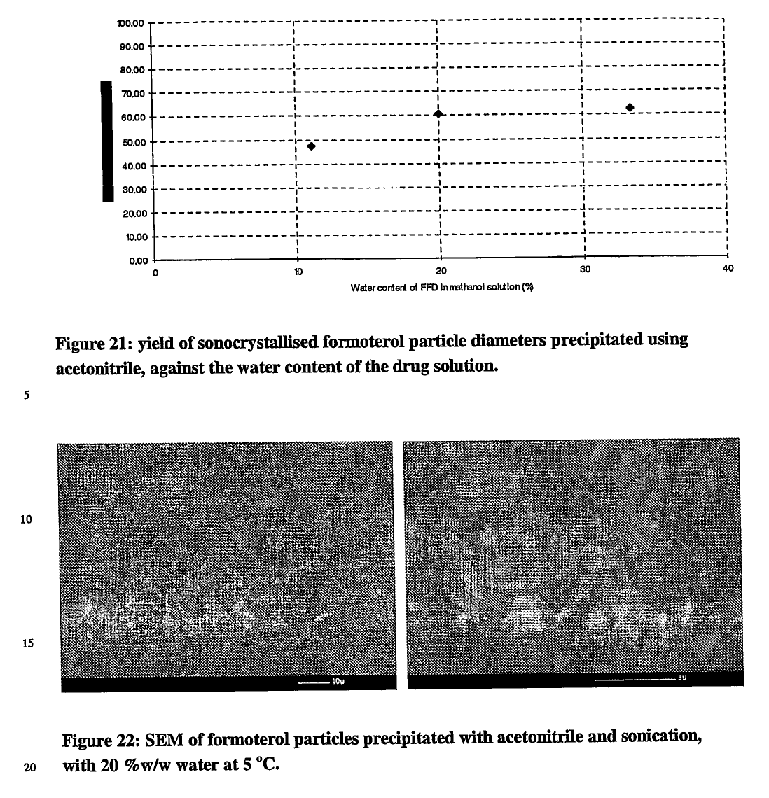

Precipitation of formoterol with both acetonitrile and diethyl ether in FIGS. 19 to 22 indicate that there is an optimum amount of water that can be added to aid crystallisation. Below this value, large particles are formed, whereas above this value a binodal size distribution is obtained, albeit within the desired size range.

Small particles within the ideal size range are produced. However there is a secondary peak for larger particles indicating that excess water could promote crystal growth.

The ideal amount of water content resulting in the smallest sized particles of formoterol is 30% w/w for diethyl ether, and 20% w/w for acetonitrile.

With regards to the yield of formoterol precipitated, the maximum achieved using diethyl ether as a precipitant was above 80% w/w, and for acetonitrile above 60% w/w. For the latter, a plateau is achieved as demonstrated on FIG. 20. However, for the highest concentration, a sharp drop in yield occurs, probably due to the low miscibility of water with diethyl ether.

Although the yield of formoterol precipitated is lower with acetonitrile, the particle diameter is undoubtedly smaller. Hence acetonitrile is the preferred precipitant for smaller particles with a d_{v(0.9)} less than 12 μm.

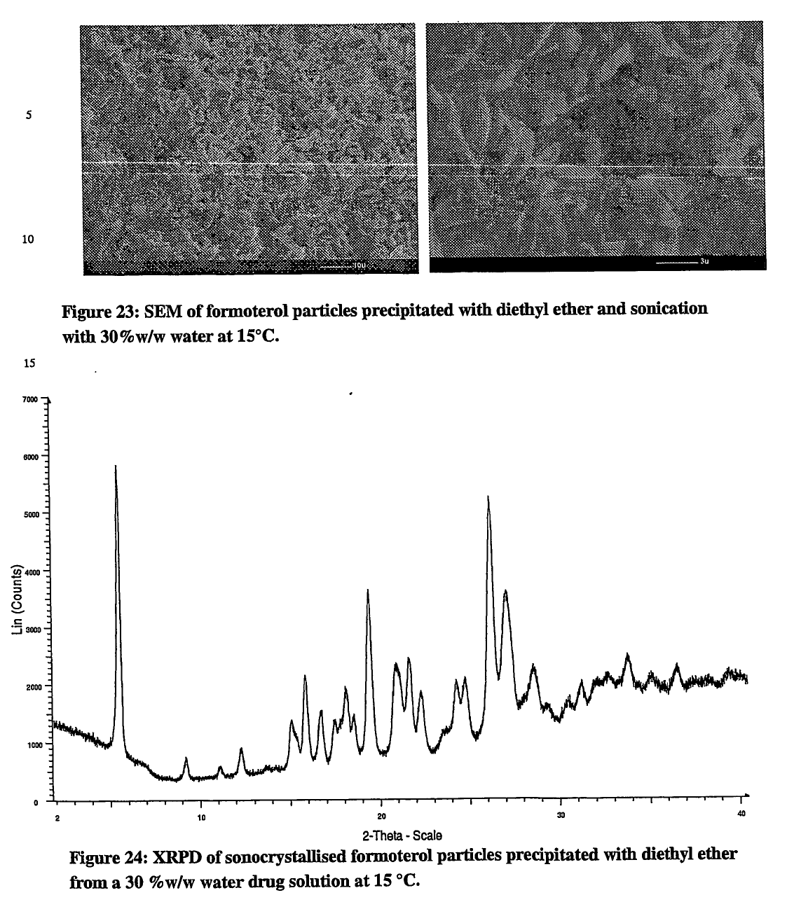

SEM analysis of the samples precipitated using both acetonitrile and diethyl ether (FIGS. 23 and 24) indicate that the crystal shapes for both samples are fairly similar. However, those produced using acetonitrile are longer and needle-like.

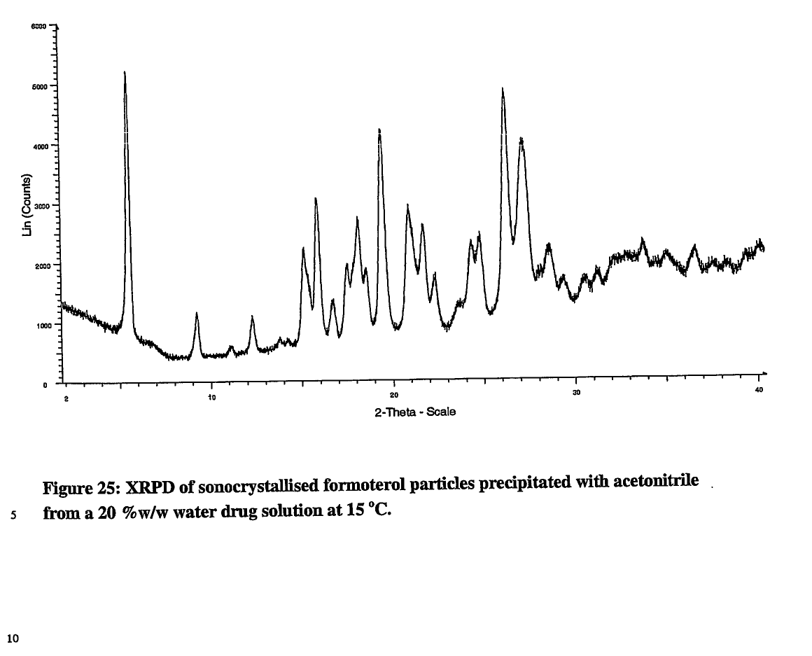

The XRPD data (FIGS. 25 and 26) for the smallest particle obtained using acetonitrile and diethyl ether are presented. They confirm that the particles obtained using both precipitants results in the formation of similar crystals. This adds a further advantage to the process, that the type of solvent being used does not affect the crystallinity of the sonocrystallised sample.

### 5.9 Example 9

### Influence of Freeze-Drying on Sonocrystallised Samples

For experimental details see example 6 with the following parameters (table 17).

TABLE 17
parameters for the study of the influence of
freeze-drying on sonocrystallised particles.

| Conditions | | |
|---|---|---|
| Solution | | 15 ml saturated budesonide in |
| | | methanol |
| Volume of precipitant | | 30 ml water |
| Temperature | | 15° C. |
| Time | | 15 minutes |
| Filter | | 0.1 μm PVDF durapore filters |
| Agitation | | Speed 6 |
| Sonic energy | Amplitude | 100% |
| | Cycle | 0.75 |

The following results were obtained (table 18). In the first set of condition (sampling of drug suspension) the particles are sized after filtration with no further drying. In the second set the particles are filtered, and freeze dried to remove traces of solvent then sized.

TABLE 18
influence of freeze drying on the particle diameters of budesonide
sonocrystallised at 15° C. from a saturated methanol solution.

| | Diameters | | |
|---|---|---|---|
| | (μm) | | |
| Condition | d_{v(0.1)} | d_{v(0.5}) | d_{v(0.9)} |
| Filtration | 1.81 | 2.88 | 4.64 |
| Filtration and freeze-drying | 1.70 | 2.80 | 4.71 |

The results demonstrate that although there is a slight change in the particle diameters with freeze drying, this is negligible. It can be concluded that filtration followed by freeze-drying has a negligible effect on particle size.

### 6 REFERENCES

1—Albert R. E., Lippmann M., Yeates D. B. Deposition, retention, and clearance of inhaled particles, Brit. J. Ind. Med. 1980, 37, 337-362.
2—Ruch P., Matijević E. Preparation of micrometer sized budesonide particles by precipitation. J. Colloid and Interface Sci. 2000, 229, 207-211.
3—Cains P. W., McCausland L. J. Sonocrystallisation—ultrasonically promoted crystallisation for the optimal isolation of drug actives. Drug Del. Sys. & Sci. 2002, 2, 47-51.
4—Kelly D. R., Harrison S. J., Jones S., Masood M. A., Morgan J. J. G. Rapid crystallisation using ultrasonic irradiation—sonocrystallisation. Tetrahedron Letters 1993, 34 (16), 2689-2690.
5—Cains P. W., McCausland L. J. Crystallisation with ultrasound. Ind. Pharm. 2002, 25, 12-13.

1. A process for producing micron-size crystalline particles of a drug substance which comprises mixing a solution of a drug substance to with a non-solvent in a container in the presence of ultrasonic energy.

2. A process according to claim 1 in which the drug substance is a hydrophilic drug.

3. A process according to claim 1 in which the solution comprises a small chain alcohol that is a solvent for a hydrophilic drugs substance.

4. A process according to claim 1 in which the solution comprises methanol.

5. A process according to claim 1 in which the non-solvent for a hydrophilic drug substance is acetonitrile, 1,1,2,2 tetrafluoroethyl 2,2,2 trifluoroethylether, diethyl ether, acetone, ethyl acetate.

6. A process according to claim 1 in which the non-solvent for a hydrophilic drugs substance is diethyl ether or acetonitrile.

7. A process according to claim 1 in which the drug substance is a hydrophobic drug.

8. A process according to claim 1 in which the solution comprises a solvent that is a small chain alcohol or chloroform.

9. A process according to claim 8 in which the solution comprises a solvent that is methanol or chloroform.

10. A process according to claim 1 in which the non-solvent for a hydrophobic drug substance is acetonitrile or water.

11. A process according to claim 1 in which the non-solvent for a hydrophobic drug substance is water.

12. A process according to claim 1 in which the drug substance is selected from mometasone, ipratropium bromide, tiotropium and salts thereof, salmeterol, fluticasone propionate, beclomethasone dipropionate, reproterol, clenbuterol, rofleponide and salts, nedocromil, sodium cromoglycate, flunisolide, budesonide, formoterol fumarate dihydrate, Symbicort® (budesonide and formoterol fumarate dihydrate), terbutaline, terbutaline sulphate and base, salbutamol base and sulphate, fenoterol, 3-[2-(4-Hydroxy-2-oxo-3H-1,3-benzothiazol-7yl)ethylamino]-N-[2-[2-(4-methylphenyl)ethoxy]ethyl]propane sulphonamide, or hydrochloride.

13. A process according to claim 1 in which the solution also contains water.

14. A process according to claim 1 in which the ultrasonic energy has a frequency of 20 kHz or more.

15. A process according to claim 1 in which the ultrasonic energy has an amplitude of between 12-260 μm.

16. A process according to claim 1 in which the burst rate of the ultrasonic energy is from 10% to 100% per second.

17. A process according to claim 1 in which the reaction temperature is between 5 and 25° C.

18. A drug substance prepared according to a process as defined in claim 1.

19. A drug substance according to claim 18 which is mometasone, ipratropium bromide, tiotropium and salts thereof, salmeterol, fluticasone propionate, beclomethasone dipropionate, reproterol, clenbuterol, rofleponide and salts, nedocromil, sodium cromoglycate, flunisolide, budesonide, formoterol fumarate dihydrate, Symbicort® (budesonide and formoterol fumarate dihydrate), terbutaline, terbutaline sulphate and base, salbutamol base and sulphate, fenoterol, 3-[2-(4-Hydroxy-2-oxo-3H-1,3-benzothiazol-7yl)ethylamino]-N-[2-[2-(4-methylphenyl)ethoxy]ethyl]propane sulphonamide, or hydrochloride.

20. A drug substance according to claim 18 having a particle size of 1 to 10 μm.
